(19) United States
(12) Patent Application Publication
(10) Pub. No.: US 2022/0105355 A1
(43) Pub. Date: Apr. 7, 2022

(54) MAGNETIZED CELL AND METHOD FOR GUIDING MAGNETIZED CELL

(71) Applicant: Hiroshima University, Hiroshima (JP)

(72) Inventors: Naosuke KAMEI, Hiroshima (JP); Mitsuo OCHI, Hiroshima (JP); Yoshikazu TANAKA, Hiroshima (JP); Naotaka HIRAMI, Hiroshima (JP)

(21) Appl. No.: 17/481,974

(22) Filed: Sep. 22, 2021

(30) Foreign Application Priority Data
Oct. 1, 2020 (JP) 2020-167006

(51) Int. Cl. A61N 2/02; A61K 41/00; A61K 35/28

(52) U.S. Cl. CPC A61N 2/02; A61K 35/28; A61K 41/00

(57) ABSTRACT

Provided is a technique that makes it possible to efficiently guide a magnetized cell by application of a magnetic field. The magnetized cell contains iron oxide, the magnetized cell containing iron, derived from the iron oxide, in an amount of not less than 35 pg/cell.

This Nonprovisional application claims priority under 35 U.S.C. § 119 on Patent Application No. 2020-167006 filed in Japan on Oct. 1, 2020, the entire contents of which are hereby incorporated by reference.

### TECHNICAL FIELD

The present invention relates to a magnetized cell that can be guided by application thereto of a magnetic field, and to a method for guiding the magnetized cell.

### BACKGROUND ART

There has been recently developed a technique for, for example, rehabilitating an injury on a specific site in the body of a patient by applying a magnetic field to magnetized cells, obtained by combination of cells and magnetic particles, so as to accumulate the magnetized cells at the specific site. Such a technique is also referred to as magnetic targeting. For example, Patent Literature 1 and Patent Literature 2 each disclose a magnetic field guidance device that can be used for magnetic targeting.

### CITATION LIST

### Patent Literatures

[Patent Literature 1]
Japanese Patent Application Publication Tokukai No. 2007-151605
[Patent Literature 2]
Japanese Patent Application Publication Tokukai No. 2020-039557

### SUMMARY OF INVENTION

### Technical Problem

However, it is unclear under what condition to magnetize a cell in order to allow the magnetized cell to be efficiently guided to and retained at a desired position by application of a magnetic field.

An aspect of the present invention has an object to provide a technique that makes it possible to efficiently guide and retain a magnetized cell by application of a magnetic field.

### Solution to Problem

In order to attain the object, a magnetized cell in accordance with an aspect of the present invention contains iron oxide, the magnetized cell containing iron, derived from the iron oxide, in an amount of not less than 35 pg/cell.

In order to attain the object, a method for guiding a magnetized cell in accordance with an aspect of the present invention includes a step of applying a magnetic field, having a magnetic flux density of not less than 0.1 T, to the magnetized cell so as to guide the magnetized cell to a desired position and retain the magnetized cell at the desired position, the magnetized cell containing iron oxide, the magnetized cell containing iron, derived from the iron oxide, in an amount of not less than 35 pg/cell.

### Advantageous Effects of Invention

According to an aspect of the present invention, it is possible to provide a technique that makes it possible to efficiently guide a magnetized cell by application of a magnetic field.

### BRIEF DESCRIPTION OF DRAWINGS

FIG. 1 is a view illustrating an example of a method for using a magnetized cell in accordance with an embodiment to rehabilitate a knee cartilage injury.

FIG. 2 is a view illustrating a state before a magnetic field is applied to magnetized cells in accordance with Examples 1 to 4.

FIG. 3 is a view illustrating a state in which the magnetized cells in accordance with Examples 1 to 4 have reached their respective stationary states by application thereto of a magnetic field.

FIG. 4 is a view showing respective guidance speeds of magnetized cells in accordance with Example 5 and Comparative Examples 1 and 2, the guidance speeds being obtained during application of a magnetic field to the magnetized cells.

### DESCRIPTION OF EMBODIMENTS

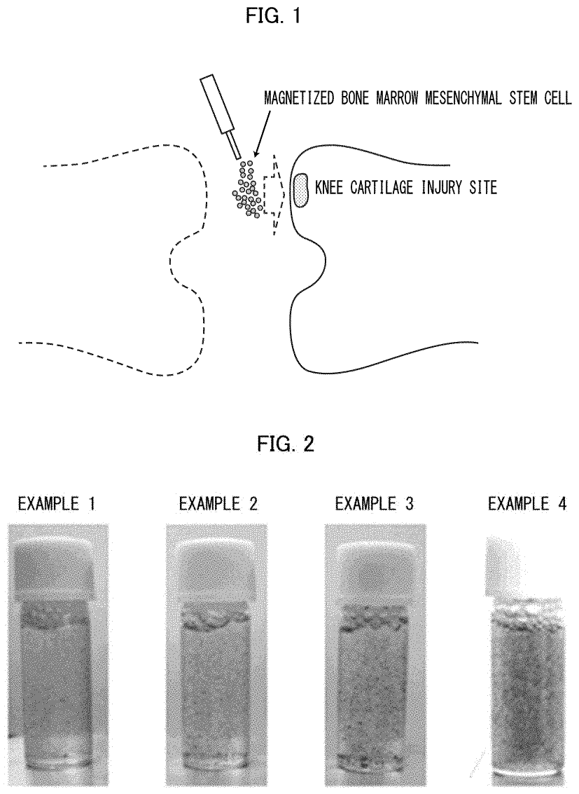

An embodiment of the present invention will be described with reference to FIG. 1. A magnetized cell in accordance with the present embodiment contains iron oxide, the magnetized cell containing iron, derived from the iron oxide, in an amount of not less than 35 pg/cell. Such a magnetized cell, which contains iron oxide, has a property such that a position of the magnetized cell is guided by action of a magnetic field. An animal cell containing iron oxide as described earlier is herein referred to as a “magnetized cell”.

(Magnetized Cell)

The magnetized cell contains iron, derived from iron oxide, in an amount of not less than 35 pg/cell. Conventionally, there has been no detailed knowledge of what magnetized cell can be guided to a desired position by application of a magnetic field. The inventors of the present invention have found the following. Specifically, only under a condition that a magnetized cell contains iron oxide containing iron in an amount of not less than 35 pg per cell, for example, a magnetic field having a magnetic flux density of 0.1 T makes it possible to guide a magnetized cell without the need to generate any excessively intense magnetic field.

The magnetized cell can contain iron, derived from iron oxide, in an amount of preferably not less than 35 pg/cell, more preferably not less than 40 pg/cell, and even more preferably not less than 45 pg/cell.

The amount of the iron contained in the magnetized cell and derived from the iron oxide has an upper limit that is not particularly limited, provided that the upper limit does not impair a function of the magnetized cell and prevents any adverse health event from occurring in an animal into which the magnetized cell has been introduced. From such a viewpoint, the amount of the iron contained in the magnetized cell and derived from the iron oxide can be, for example, not more than 1 pg/cell, not more than 500 pg/cell, not more than 250 pg/cell, not more than 200 pg/cell, not more than 150 pg/cell, or not more than 125 pg/cell.

The iron oxide contained in the magnetized cell is preferably superparamagnetic. In a case where the iron oxide is superparamagnetic and is contained in the magnetized cell in a larger amount, a guidance force caused by application of a magnetic field efficiently acts on the iron oxide. This makes it easy to guide and retain the magnetized cell by application of a magnetic field.

The iron oxide contained in the magnetized cell is preferably coated with a water-soluble polysaccharide. Examples of the water-soluble polysaccharide with which the iron oxide is coated include dextran, dextrin, cellulose, hyaluronic acid, gelatin, mannan, pullulan, and chondroitin sulfate. Among these water-soluble polysaccharides, carboxydextran is preferable. The water-soluble polysaccharide with which the iron oxide is coated can be one of these water-soluble polysaccharides, or can be a mixture of two or more of the water-soluble polysaccharides. Such a water-soluble polysaccharide makes it possible to (i) inexpensively and easily coat the iron oxide and (ii) effectively prevent an adverse effect on a cell of the iron oxide.

Specific examples of the iron oxide which is coated with such a water-soluble polysaccharide include ferucarbotran. Ferucarbotran is iron oxide particles in which maghemite (γ-Fe₂O₃) is coated with carboxydextran. Ferucarbotran is clinically used as a contrast medium for magnetic resonance imaging (MRI). In terms of its established safety for the human body, ferucarbotran is preferable as the iron oxide contained in the magnetized cell. Also in terms of its superparamagnetism, ferucarbotran is also preferable as the iron oxide contained in the magnetized cell.

The magnetized cell is preferably suspended in an infusion while being injected into the body of an animal by, for example, injection. Such an infusion is preferably an isotonic electrolyte infusion and can be, for example, physiological saline, Ringer's solution, or a glucose solution. The infusion can be one of these solutions, or can be a mixture of two or more of the solutions.

(Example of Use of Magnetized Cell)

An animal cell serving as a host for the magnetized cell is not particularly limited provided that the animal cell is an animal-derived cell. As an example of use of the magnetized cell, the following description will discuss a case where the animal cell serving as the host is a bone marrow-derived mesenchymal stem cell (hereinafter referred to as a “bone marrow MSC”).

A cartilage injury that is caused in a case where a cartilage of a joint of the human body peels off together with a surface layer of a bone located at or near the cartilage is extremely less likely to be naturally rehabilitated. In order to rehabilitate such a cartilage injury, it is effective to accumulate, in an affected part, cells having a function of regenerating a cartilage or a bone. For example, a bone marrow MSC is known as such a cell.

FIG. 1 illustrates an example of a method for rehabilitating a knee cartilage injury. Bone marrow MSCs have been conventionally known to exhibit a knee cartilage regeneration effect by being accumulated at a knee cartilage injury site. However, mere injection of the bone marrow MSCs at or near the knee cartilage injury site (affected part) is less likely to cause cartilage regeneration. This is considered to be because the injected bone marrow MSCs are dispersed in the body without being accumulated at the knee cartilage injury site.

In contrast, a magnetized cell for which a bone marrow MSC serves as a host has a property of being capable of being guided to a desired position by application of a magnetic field. Thus, as illustrated in FIG. 1, in a case where magnetized cells for which bone marrow MSCs serve as a host are injected at or near a knee cartilage injury site and a magnetic field is applied so that the magnetized cells are guided to the injury site, the magnetized cells are efficiently accumulated at the injury site. Such magnetized cells therefore make it possible to rehabilitate a knee cartilage injury with high efficiency.

The magnetized cells are guided to the injury site within a few seconds to a few minutes at the latest after the magnetic field starts to be applied. Bone marrow MSCs, for example are clinically required to be accumulated at an affected part within approximately 10 minutes. Magnetized cells in accordance with the present embodiment can be more quickly guided to and accumulated at an affected part.

Note that an animal cell serving as a host for a magnetized cell is not limited in type to a bone marrow MSC, but can be, for example, a mesenchymal stem cell that is not derived from bone marrow, or a stem cell that is different from a mesenchymal stem cell. An appropriate type of stem cell can be selected in accordance with an injury site to be rehabilitated. An application of the magnetized cell is not limited to rehabilitation of an injury in the body of an animal. For example, in order to use magnetized cells as a marker, it is possible to accumulate the magnetized cells, for example, at or near a specific organ in the body of the animal. An animal cell serving as a host for a magnetized cell can therefore be another type of cell different from a stem cell.

The animal cell can be a human cell, a non-human mammal cell, or any other animal cell different from the non-human mammal cell.

(Method for Producing Magnetized Cell)

A method for producing a magnetized cell is not particularly limited, but can be, for example, a method in which iron oxide is added in a culture medium, in which animal cells serving as a host for magnetized cells are cultured, so that the animal cells are cultured for a predetermined period of time. An appropriate amount of iron oxide to be added and an appropriate culture time can be selected as appropriate in accordance with, for example, a type of animal cells serving as a host for magnetized cells, a type of iron oxide, a type of culture medium, and/or the number of cells that are being cultured. A magnetized cell is obtained in a case where an animal cell incorporates such iron oxide into a cell by a mechanism such as endocytosis.

(Method for Guiding Magnetized Cell)

A method for guiding a magnetized cell in accordance with the present embodiment includes a step of applying a magnetic field, having a magnetic flux density of not less than 0.1 T, to the magnetized cell so as to guide the magnetized cell to a desired position and retain the magnetized cell at the desired position, the magnetized cell containing iron oxide, the magnetized cell containing iron, derived from the iron oxide, in an amount of not less than 35 pg/cell.

Application of a magnetic field having a magnetic flux density of not less than 0.1 T suffices to guide and retain the magnetized cell. The magnetic field having a magnetic flux density of not less than 0.1 T can be easily generated with use of any of various magnetic field generation sources. Examples of such a magnetic field generation source include a solenoid coil, a superconducting coil, a superconducting magnet, and a permanent magnet.

The magnetic field generation source is preferably a solenoid coil. In this case, since a magnetic field having a magnetic flux density of not less than 0.1 T only needs to be generated, it is unnecessary to apply an excessively large electric current to the solenoid coil. The solenoid coil also easily allows the magnetic field to be oriented orthogonally to an affected part. The solenoid coil can also be designed so as to allow the affected part to be inserted in a hollow part. Such a design makes it easy to adjust a position of the solenoid coil relative to the affected part. This allows a direction in which the magnetized cell is guided to be easily adjusted. For example, the solenoid coil has a center having the most intense magnetic field. Thus, in a case where the position of the solenoid coil is adjusted so that the affected part is located at or near the center, the magnetized cell is easily guided to and retained at the affected part.

In other words, the method for guiding a magnetized cell in accordance with the present embodiment is preferably configured such that, in the step, a solenoid coil is used to generate the magnetic field, the magnetized cell is injected at or near an affected part of an animal, and the affected part is located at or near a center of the solenoid coil. Note that a magnetic field generation source including a solenoid coil can be, for example, a magnetic field guidance device disclosed in Patent Literature 1 or Patent Literature 2.

The magnetic field that is applied to the magnetized cell has a magnetic flux density of preferably not less than 0.1 T, more preferably not less than 0.15 T, and even more preferably not less than 0.2 T. The magnetized cell to which a more intense magnetic field is applied is easily guided and retained. However, in a case where the solenoid coil or the like is used as the magnetic field generation source, it is necessary to apply thereto a large electric current in order to generate an intense magnetic field. In order to prevent electric power that is more than necessary from being consumed, the magnetic field that is applied to the magnetized cell can have a magnetic flux density of not more than 1 T, preferably not more than 0.5 T, more preferably not more than 0.3 T, and even more preferably not more than 0.2 T.

(Kit for Guiding Magnetized Cell)

A kit for guiding a magnetized cell in accordance with the present embodiment includes a magnetized cell containing iron oxide, the magnetized cell containing iron, derived from the iron oxide, in an amount of not less than 35 pg/cell. For a configuration of the magnetized cell, the above description can be referred to.

Alternatively, the kit for guiding a magnetized cell in accordance with the present embodiment can include (i) an iron oxide-free animal cell instead of the magnetized cell (described earlier) and (ii) iron oxide.

Such an animal cell is not particularly limited provided that the animal cell is an animal-derived cell. The animal cell can be, for example, a bone marrow MSC, a mesenchymal stem cell that is not derived from bone marrow, or a stem cell that is different from a mesenchymal stem cell. An appropriate type of stem cell can be selected in accordance with an injury site to be rehabilitated. Alternatively, the animal cell can be another type of cell different from a stem cell. The animal cell can be a human cell, a non-human mammal cell, or any other animal cell different from the non-human mammal cell.

Iron oxide can be incorporated into the animal cell so that iron derived from the iron oxide is contained in the magnetized cell in an amount of not less than 35 pg/cell. The iron oxide is preferably superparamagnetic or ferromagnetic. The iron oxide is also preferably coated with a water-soluble polysaccharide such as dextran. Preferable examples of such iron oxide include ferucarbotran.

The kit for guiding a magnetized cell in accordance with the present embodiment can further include a magnetic field generation source that is capable of generating a magnetic field having a magnetic flux density of not less than 0.1 T. Examples of such a magnetic field generation source include a solenoid coil, a superconducting coil, a superconducting magnet, and a permanent magnet. The magnetic field generation source is preferably a solenoid coil. A magnetic field generation source including a solenoid coil can be, for example, a magnetic field guidance device disclosed in Patent Literature 1 or Patent Literature 2.

The kit for guiding a magnetized cell in accordance with the present embodiment can further include a reagent such as an animal cell culture medium, an instrument such as a syringe, a device such as a stabilized DC power supply for supplying an electric current to the solenoid coil, and/or others such as an instruction manual.

Aspects of the present invention can also be expressed as follows:

A magnetized cell in accordance with an aspect of the present invention contains iron oxide, the magnetized cell containing iron, derived from the iron oxide, in an amount of not less than 35 pg/cell. With the configuration, application of a magnetic field allows the magnetized cell to be guided to and retained at a desired position.

The magnetized cell in accordance with an aspect of the present invention can be configured such that the iron derived from the iron oxide is contained in an amount of not more than 125 pg/cell. With the configuration, it is possible to easily produce a magnetized cell containing iron oxide in such an amount.

A method for guiding a magnetized cell in accordance with an aspect of the present invention includes a step of applying a magnetic field, having a magnetic flux density of not less than 0.1 T, to the magnetized cell so as to guide the magnetized cell to a desired position and retain the magnetized cell at the desired position, the magnetized cell containing iron oxide, the magnetized cell containing iron, derived from the iron oxide, in an amount of not less than 35 pg/cell.

The method for guiding a magnetized cell in accordance with an aspect of the present invention can be configured such that, in the step, a solenoid coil is used to generate the magnetic field, the magnetized cell is injected at or near an affected part of an animal, and the affected part is located at or near a center of the solenoid coil.

### Examples

[1. Guidance of Magnetized Cell by Magnetic Field]

A magnetized cell in accordance with an example of the present invention was subjected to an experiment in which the magnetized cell is guided by application thereto of a magnetic field having a magnetic flux density of approximately 0.1 T.

### 1-1. Experimental Conditions

A solenoid coil was used to generate the magnetic field applied to the magnetized cell. The solenoid coil had an inside diameter of 200 mm, an outside diameter of 300 mm, a coil diameter of 2 mm, 100 axial stages, 20 radial stages, 2000 turns in total, and an axial length of 200 mm. Application of an electric current of 12 A to the solenoid coil causes a magnetic field having a magnetic flux density of approximately 0.1 T to be generated at a coil center at an end of the solenoid coil.

A bone marrow MSC into which ferucarbotran had been incorporated was prepared as the magnetized cell. The ferucarbotran was incorporated into the bone marrow MSC by adding ferucarbotran in a culture medium, in which bone marrow MSCs were cultured, so as to culture the bone marrow MSCs. Table 1 below shows the iron content (Fe content) per cell obtained after the culture.

The iron content per unit volume is an average of results obtained by dividing each sample into three equal parts and using an inductively coupled plasma (ICP) emission analyzer to subject the parts to measurement. The iron content per cell was found by (i) calculating the iron content in the sample as a whole (whole cells) from the iron content per unit volume and (ii) dividing, by the number of cells in the sample, the iron content calculated in (i).

TABLE 1

| | | Fe content | Fe content | Fe content |
|---|---|---|---|---|
| | Number of | per unit volume | in whole | per cell |
| Sample | cells | (μg/mL) | cells (μg) | (pg/cell) |
| Example 1 | 5.00E+05 | 0.2519 | 25.2 | 50.4 |
| Example 2 | 5.00E+05 | 0.4113 | 41.2 | 82.4 |
| Example 3 | 1.20E+06 | 0.5444 | 54.4 | 45.3 |
| Example 4 | 2.90E+06 | 3.5587 | 355.9 | 123.0 |

### 1-2. Experimental Results

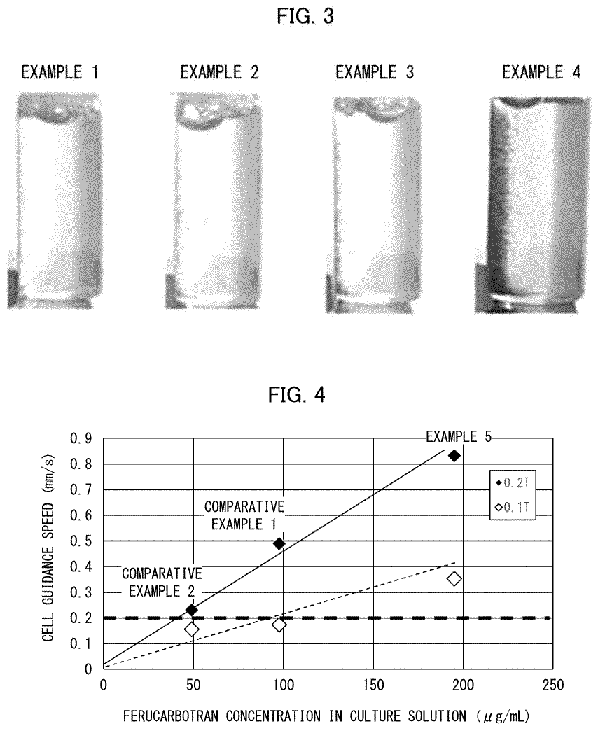

The following description will discuss experimental results with reference to FIGS. 2 and 3. FIG. 2 illustrates a state before a magnetic field is applied to magnetized cells of Examples 1 to 4. FIG. 3 illustrates a state in which the magnetized cells of Examples 1 to 4 have reached their respective stationary states by application thereto of a magnetic field having a magnetic flux density of approximately 0.1 T. Note that the present experiment was carried out while each magnetized cell sample was suspended in physiological saline.

As shown in FIGS. 2 and 3, the magnetized cells were substantially uniformly suspended in the physiological saline before a magnetic field was applied thereto. In contrast, the magnetized cells to which a magnetic field having a magnetic flux density of approximately 0.1 T was applied by the solenoid coil were guided in the axial direction of the solenoid coil. The above results show that a magnetized cell in accordance with an embodiment of the present invention can be guided by a magnetic field having a magnetic flux density of not less than 0.1 T.

[2. Speed at which Magnetized Cell is Guided by Magnetic Field]

Next, a magnetized cell in accordance with an example of the present invention or a magnetized cell in accordance with a comparative example was subjected to an experiment in which a speed (guidance speed) at which the magnetized cell is moved by guidance by a magnetic field was measured by application, to the magnetized cell, of a magnetic field having a magnetic flux density of approximately 0.1 T or approximately 0.2 T.

### 2-1. Experimental Conditions

Magnetized cells of Example 5 and Comparative Examples 1 and 2 were prepared by (i) adding ferucarbotran in a culture medium in which bone marrow MSCs were cultured and (ii) culturing the bone marrow MSCs for 12 hours. Table 2 below shows (i) a concentration of ferucarbotran added in the culture medium and (ii) the iron content in a resultant cell.

TABLE 2

| | Ferucarbotran | |
|---|---|---|
| | concentration | Fe content per cell |
| Sample | (μg/mL) | (pg/cell) |
| Example 5 | 195 | 53 |
| Comparative | 97.6 | 28 |
| Example 1 | | |
| Comparative | 49 | 14 |
| Example 2 | | |

The magnetized cells of Example 5 and Comparative Examples 1 and 2 were each dissolved in physiological saline and allowed to stand in a water channel having a width of 2 mm. Thereafter, a magnetic field having a magnetic flux density of approximately 0.1 T or approximately 0.2 T was applied to these cells so that respective guidance speeds of the cells were measured.

A solenoid coil was used to generate the magnetic field applied to the cells. The solenoid coil had an inside diameter of 240 mm, an outside diameter of 404 mm, a width of 119 mm, 29 axial stages, 25 radial stages, and 725 turns in total. Application of an electric current of 33 A to the solenoid coil causes a magnetic field having a magnetic flux density of approximately 0.1 T to be generated at a coil center at an end of the solenoid coil. Furthermore, application of an electric current of 66 A to the solenoid coil causes a magnetic field having a magnetic flux density of approximately 0.2 T to be generated at the coil center at the end of the solenoid coil.

The solenoid coil was located so that the water channel extends along a straight line including the central axis of the solenoid coil. Note that the solenoid coil is designed such that a magnetic field having a magnetic flux density of approximately 0.1 T or approximately 0.2 T is stably applied in a range in which the cells are guided in the water channel.

### 2-2. Experimental Results

The following description will discuss experimental results with reference to FIG. 4. FIG. 4 shows results of measurement of guidance speeds at which cells of Example 5 and Comparative Examples 1 and 2 are guided in the axial direction of the solenoid coil in a case where a magnetic field having a magnetic flux density of approximately 0.1 T or approximately 0.2 T is applied to the cells. Note that the cells in the physiological saline to which cells no magnetic field was applied were sedimented at a speed of 0.1 mm/s to 0.2 mm/s. It has therefore been determined that a guidance effect was brought about by application of the magnetic field in a case where a “cell guidance speed” was more than 0.2 mm/s.

As shown in FIG. 4, the magnetized cell of Example 5 to which a magnetic field having a magnetic flux density of not less than 0.1 T was applied was observed to exhibit a guidance effect brought about by application of the magnetic field. In contrast, the magnetized cells of Comparative Examples 1 and 2 to which a magnetic field having a magnetic flux density of approximately 0.2 T was applied were observed to exhibit the guidance effect, whereas the magnetized cells of Comparative Examples 1 and 2 to which a magnetic field having a magnetic flux density of approximately 0.1 T was applied were observed to exhibit no guidance effect. Results obtained from a group to which a magnetic field having a magnetic flux density of approximately 0.1 T was applied have suggested that a magnetized cell containing iron in an amount of approximately not less than 30 pg/cell exhibits a guidance speed of more than 0.2 mm/s. This shows that a magnetized cell containing iron in an amount of not less than 35 pg/cell can be stably guided by a magnetic field having a magnetic flux density of not less than 0.1 T.

### Additional Remarks

The present invention is not limited to the embodiments or examples, but can be altered by a skilled person in the art within the scope of the claims. The present invention also encompasses, in its technical scope, any embodiment derived by combining technical means disclosed in differing embodiments or examples.

### INDUSTRIAL APPLICABILITY

The present invention can be used for animal regenerative medicine, for example.

1. A magnetized cell comprising iron oxide,
the magnetized cell containing iron, derived from the iron oxide, in an amount of not less than 35 pg/cell.

2. The magnetized cell as set forth in claim 1, wherein the iron derived from the iron oxide is contained in an amount of not more than 125 pg/cell.

3. A method for guiding a magnetized cell, comprising a step of applying a magnetic field, having a magnetic flux density of not less than 0.1 T, to the magnetized cell so as to guide the magnetized cell to a desired position, the magnetized cell containing iron oxide, the magnetized cell containing iron, derived from the iron oxide, in an amount of not less than 35 pg/cell.

4. The method as set forth in claim 3, wherein, in the step,
a solenoid coil is used to generate the magnetic field,
the magnetized cell is injected at or near an affected part of an animal, and
the affected part is located at or near a center of the solenoid coil.
